(19) United States
(12) Patent Application Publication
(10) Pub. No.: US 2024/0268809 A1
(43) Pub. Date: Aug. 15, 2024

(54) TISSUE REPAIR CONSTRUCTS AND METHODS OF USE

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventor: Mark E. Housman, North Attleboro, MA (US)

(73) Assignee: Smith & Nephew, Inc., Memphis, TN (US)

(21) Appl. No.: 18/414,781

(22) Filed: Jan. 17, 2024

Related U.S. Application Data
(60) Provisional application No. 63/484,634, filed on Feb. 13, 2023.

(51) Int. Cl. A61B 17/04; A61B 17/00

(52) U.S. Cl. CPC A61B 17/04; A61B 2017/00831; A61B 2017/00982

(57) ABSTRACT

Knotless soft suture constructs include a repair tape having a finger trap. The repair tape is preloaded with a transfer suture through the finger trap. The transfer suture is used to capture an end of the repair tape through itself, forming a cinching suture loop that can be tightened around the repair but not loosened.

### CROSS-REFERENCE TO RELATED APPLICATIONS

This application claims priority to and benefit of U.S. Provisional Application No. 63/484,634, filed on Feb. 13, 2023, entitled TISSUE REPAIR CONSTRUCTS AND METHODS OF USE, the entire contents of which are incorporated herein by reference for all purposes.

### FIELD

The present disclosure relates to surgical repair of tissue and, more specifically, to knotless tissue repair constructs for affixing a tissue graft to bone.

### BACKGROUND

Surgical tissue repairs, such as a rotator cuff repair, are often accomplished by passing sutures through transosseous tunnels in bone. A surgeon may secure the suture to the tunnels using tacks, staples, knotted suture anchors and/or knotless suture anchors, and then use the suture to secure the tissue or tissue graft to bone. In general, surgeons prefer knotless suture anchor constructs because the knot does not interfere with the repair. However, such suture anchor constructs may not allow for controlled tensioning of the suture attached to the anchor. Surgeons may also find it desirable to reduce the amount of material implanted in a tissue repair.

### SUMMARY

The disclosure describes a knotless soft suture construct for completing a tissue repair, such as a rotator cuff repair. The construct consists of a repair tape having a finger trap. The repair tape is preloaded with a transfer suture through the finger trap. The transfer suture is used to capture an end of the repair tape through itself, forming a cinching suture loop that can be tightened around the repair but not loosened. Advantageously, the construct of this disclosure both eliminates knots and allows for controlled tensioning of the repair construct. It may also reduce cost of completion, as well as the amount of material implanted compared with other types of repair constructs.

Further examples of the tissue repair constructs and methods of this disclosure may include one or more of the following, in any suitable combination.

In examples, the tissue repair construct of this disclosure includes a repair suture. The repair suture has a first section, a second section connected to the first section, and a third section connected to the second section opposite the first section. The third section has a spliced region adjacent to the second section. The construct also includes a transfer suture. A portion of the transfer suture extends through the spliced region such that first and second ends of the transfer suture extend from opposite ends of the spliced region. The spliced region forms a finger trap, such that the spliced region allows movement of the transfer suture through the spliced region in a first direction but prevents movement of the transfer suture through the spliced region in a second direction opposite the first direction.

In further examples, a material of the second section is selected to be different than a material of the first section. In examples, a material of the third section is selected to be different than a material of the first section. In examples, a material of the third section is selected to be different than a material of the second section. In examples, the first section is made of 2-0 suture. In examples, the second section is made of suture tape. In examples, the third section is made of No. 2 suture. In examples, the first end of the transfer suture includes a transfer loop.

In other examples, the tissue repair construct of this disclosure includes a repair suture. The repair suture has a first section, a second section connected to the first section, and a third section connected to the second section opposite the first section. The third section has a spliced region adjacent to the second section. The first section and a portion of the second section extends through the spliced region, forming a repair loop. The spliced region forms a finger trap, such that the repair loop can be tightened around a repair but not loosened.

In further examples, a material of the second section is selected to be different than a material of the first section. In examples, a material of the third section is selected to be different than a material of the first section. In examples, a material of the third section is selected to be different than a material of the second section. In examples, the first section is made of 2-0 suture. In examples, the second section is made of suture tape. In examples, the third section is made of No. 2 suture.

In examples, a method of tissue repair of this disclosure includes forming a tunnel through a bone adjacent a tissue in need of repair. A tissue repair construct is passed through the tunnel and through the tissue such that a first end of a third section and a second end of a transfer suture of the tissue repair construct extend from the tunnel, and a first section and a first end of the transfer suture of the tissue repair construct extend from of the tissue. The first end of the transfer suture includes a transfer loop. The tissue repair construct further includes a repair suture. The repair suture has the first section, a second section connected to the first section, and the third section connected to the second section opposite the first section. The third section has a spliced region adjacent to the second section. A portion of the transfer suture extends through the spliced region such that the first and second ends of the transfer suture extend from opposite ends of the spliced region. The method also includes passing the first section of the tissue repair construct through the transfer loop. Using the second end of the transfer suture, the first section and a portion of the second section are pulled through the spliced region to form a repair loop extending along a surface of the tissue and the bone. The repair loop is tensioned to secure the tissue against the bone. The spliced region forms a finger trap, such that the repair loop can be tightened along the surface of the tissue and the bone, but not loosened. In further examples, the bone is a humeral head, and the tissue is a rotator cuff. In examples, the transfer suture is preassembled to the repair suture. In examples, the second section is made of suture tape.

A reading of the following detailed description and a review of the associated drawings will make apparent the advantages of these and other features. Both the foregoing general description and the following detailed description serve as an explanation only and do not restrict aspects of the disclosure as claimed.

### BRIEF DESCRIPTION OF THE DRAWINGS

Reference to the detailed description, combined with the following figures, will make the disclosure more fully understood, wherein:

FIG. 1 illustrates an example of a repair suture for use in a tissue repair construct of this disclosure;

FIGS. 2A and 2B illustrates the splicing of a transfer suture to the repair suture of FIG. 1;

FIG. 3 illustrates an example of the tissue repair construct of this disclosure; and

FIGS. 4A-F illustrate a method of tissue repair using the tissue repair construct of FIG. 3.

### DETAILED DESCRIPTION

In the following description, like components have the same reference numerals, regardless of different illustrated examples. To illustrate examples clearly and concisely, the drawings may not necessarily reflect appropriate scale and may have certain features shown in somewhat schematic form. The disclosure may describe and/or illustrate features in one example, and in the same way or in a similar way in one or more other examples, and/or combined with or instead of the features of the other examples.

In the specification and claims, for the purposes of describing and defining the invention, the terms “about” and “substantially” represent the inherent degree of uncertainty attributed to any quantitative comparison, value, measurement, or other representation. The terms “about” and “substantially” moreover represent the degree by which a quantitative representation may vary from a stated reference without resulting in a change in the basic function of the subject matter at issue. Open-ended terms, such as “comprise,” “include,” and/or plural forms of each, include the listed parts and can include additional parts not listed, while terms such as “and/or” include one or more of the listed parts and combinations of the listed parts.

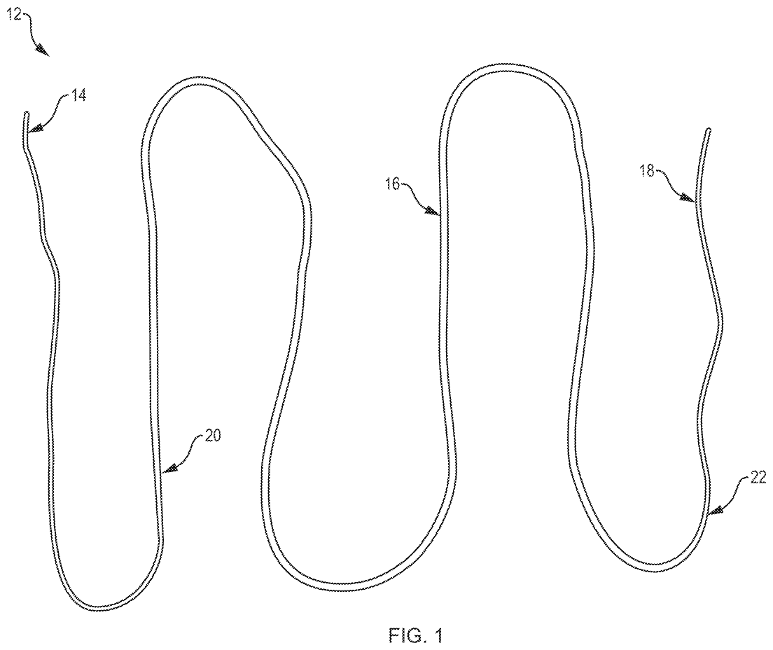

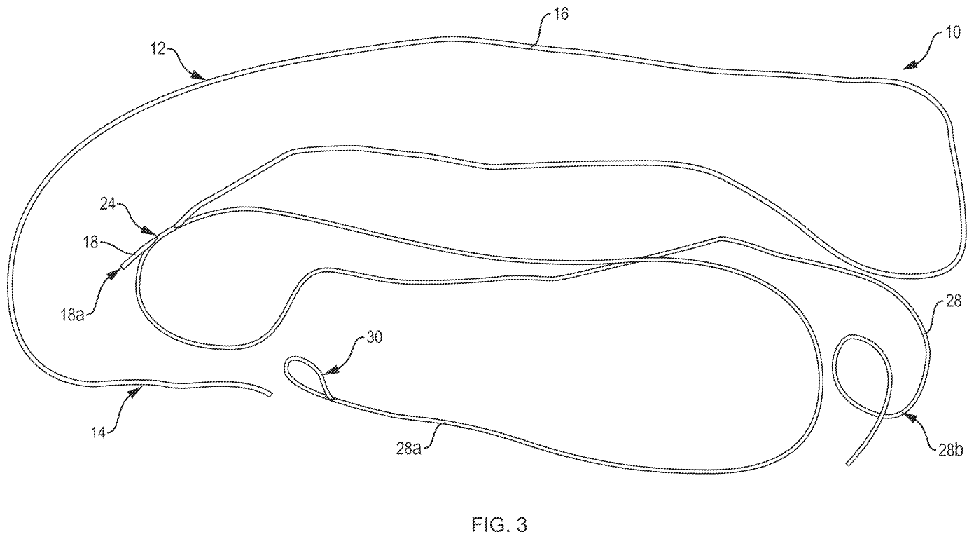

FIG. 1 illustrates an example of a repair suture 12 for use in a tissue repair construct 10 (FIG. 3) of this disclosure. As shown in FIG. 1, the repair suture 12 may comprise a first section 14, a second section 16 and a third section 18. A first transition region 20 may extend between the first section 14 and the second section 16 and a second transition region 22 may extend between the second section 16 and the third section 18. The first section 14 may comprise a small diameter suture. For example, the first section 14 may comprise a 2-0 suture. The second section 16 may comprise a flat tape, which advantageously may be less traumatic to a repair than traditional round suture. However, the disclosure contemplates that other types of suture, such as No. 2 suture, could be used in the second section 16. The third section 18 may comprise a round, cannulated suture. For example, the third section 18 may comprise No. 2 suture. The third section 18 may be used to form a spliced region, as further described below.

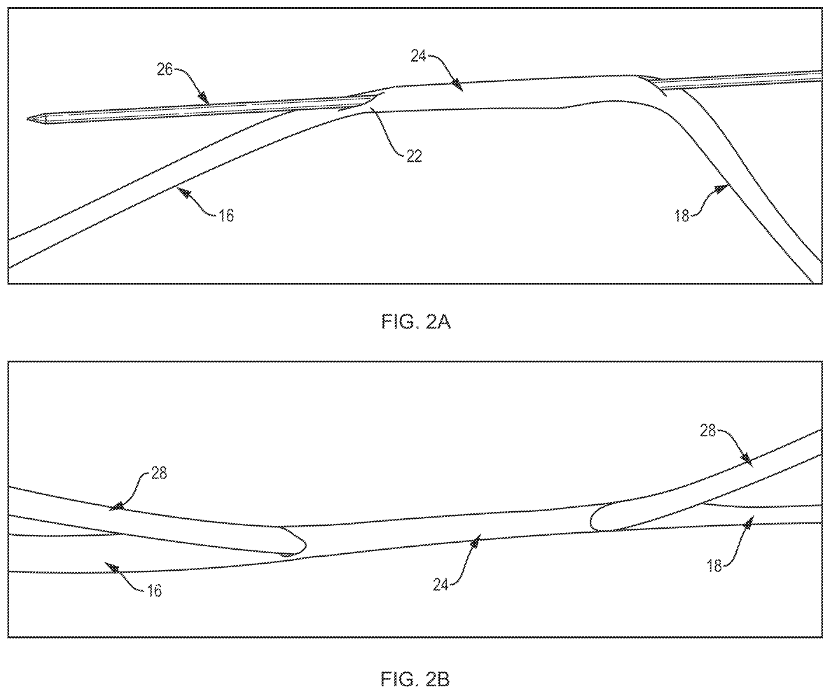

FIGS. 2A and 2B illustrates the splicing of a transfer suture 28 to the third section 18 of the tissue repair construct 10. As shown in FIG. 2A, a needle or other suitable instrument 26 may be inserted through the cannulated third section 18 adjacent the transition region 22 to form a spliced region 24. As shown in FIG. 2B, the transfer suture 28 may be loaded through the spliced region 24 such that each end of the transfer suture 28 extends from opposing sides of the spliced region 24. In examples, the transfer suture 28 may comprise a hollow braid suture made out of ultra-high-molecular-weight polyethylene that is spliced to itself to form a smooth transfer loop. However, the disclosure contemplates that the transfer suture 28 could be a Nitinol loop, or other types of sutures.

FIG. 3 illustrates an example of the tissue repair construct 10 of this disclosure. As shown in FIG. 3, the transfer suture 28 may be preassembled to the spliced region 24 in the third section 18. A first end 28a of the transfer suture 28 may include a transfer loop 30 for pulling the first section 14 and a portion of the second section 16 through the spliced region 24 by pulling on the second end 28b of the transfer suture 28, as further described below. The transfer loop 30 may be a size zero to a size 2-0 suture. The spliced region 24 may be configured to act as a “finger trap,” allowing movement of suture through the spliced region 24 in one direction but preventing movement of suture through the spliced region 24 in the opposite direction. A first end 18a of the third section 18 may form a repair tail used to control and tension the tissue repair construct 10 during use.

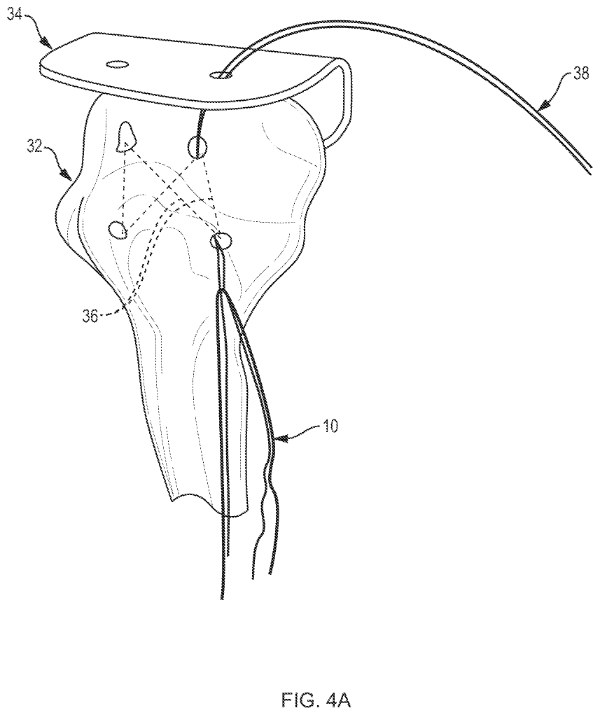

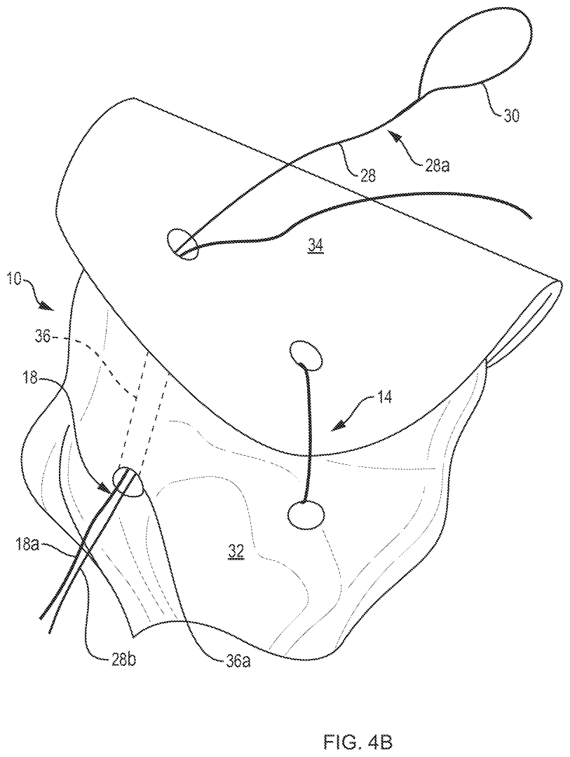

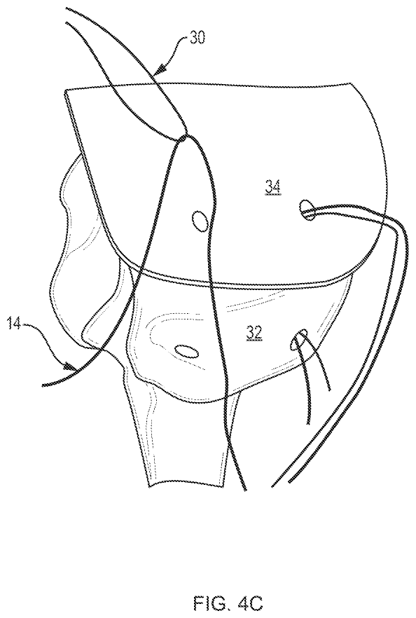

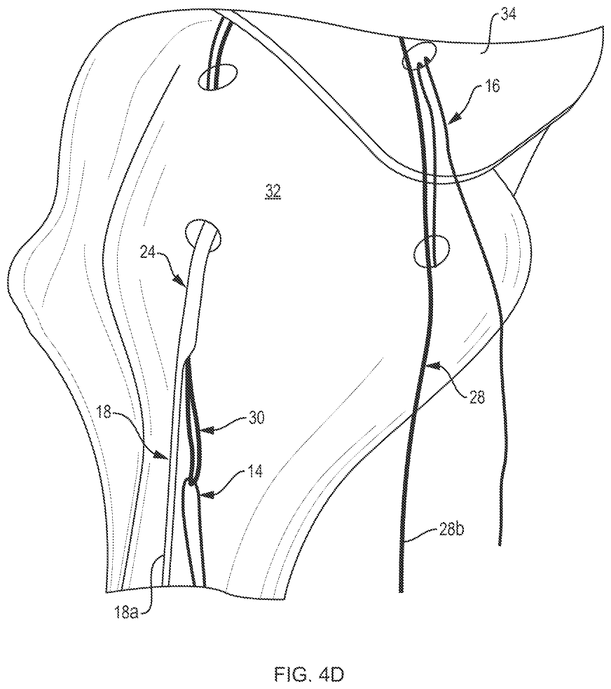

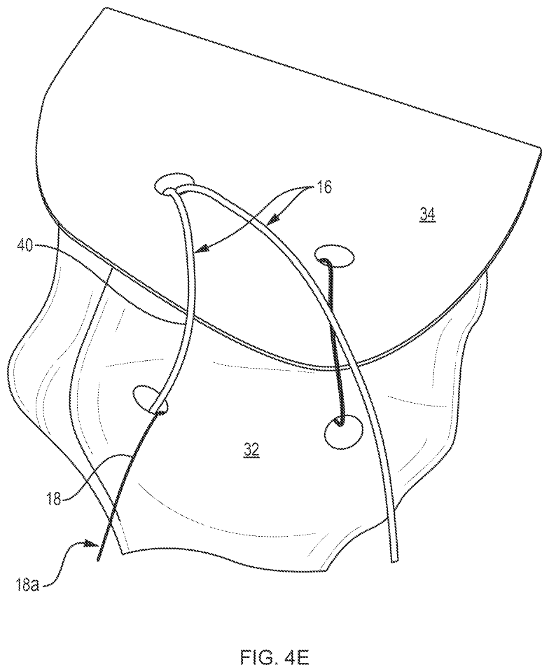

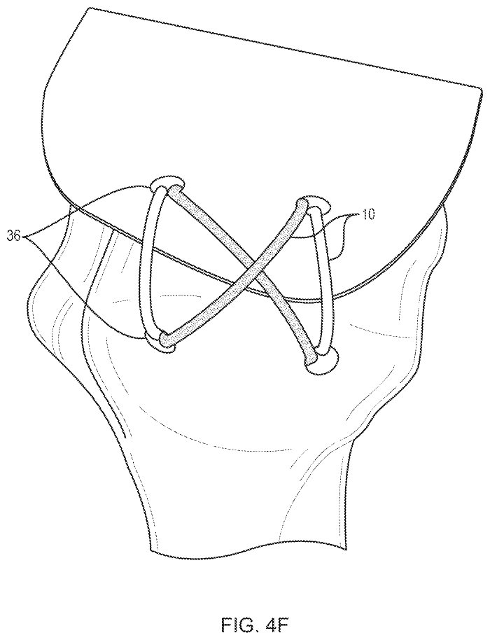

FIGS. 4A-F illustrate a method of tissue repair, such as rotator cuff repair, using one or more tissue repair constructs 10 of this disclosure. As shown in FIG. 4A, the repair site may include a bone 32, such as a humeral head, and a tissue 34 in need of repair, such as a torn rotator cuff. To begin the repair, a user may drill one or more bone tunnels 36 through the bone 32. For example, the surgeon may drill four tunnels 36, as shown. The user may then use a suture passing device 38, such as a monofilament loop, to pull a first tissue repair construct 10 through one of the bone tunnels 36 and through the tissue 34. As shown in FIG. 4B, pulling the tissue repair construct 10 through the bone tunnel 36 exposes the first end 18a of the third section 18 and the second end 28b of the transfer suture 28 outside of the bone 32. This action also exposes the first section 14 of the tissue repair construct 10 and the first end 28a of the transfer suture 28 outside of the tissue 34. In the example of FIG. 4B, the spliced region 24 of the tissue repair construct 10 may be placed just beneath the surface of the bone 32 inside the lateral end 36a of the tunnel 36. However, the spliced region 24 may also be positioned outside of the tunnel 36 (FIG. 4D). In this way, the tissue repair construct 10 may be adjusted in the tunnel 36 according to user preference.

As shown in FIG. 4C, the user then passes the first section 14 of the tissue repair construct 10 through the transfer loop 30. As shown in FIG. 4D, while holding the repair construct 10 in place with the first end 18a of the third section 18, the user then uses the second end 28b of the transfer suture 28 to pull the first section 14 and a portion of the second section 16 through the spliced region 24 to form a cinching repair loop 40 around and through the tissue 34 (FIG. 4E). In this way, the portion of the tissue repair construct 10 that affixes soft tissue to bone may be the atraumatic flat second section 16, rather than a round suture. The repair loop 40 may bring the tissue 34 in contact with the bone 32 when the repair construct 10 is subsequently tensioned by the user. Notably, the smaller diameter of the first section 14 and the transfer suture 28 allow the second section 16 to be pulled through the spliced region 24 without an excessively high pull-through load.

As shown in FIG. 4E, with the second section 16 having been feed through itself, and with the repair construct 10 now forming the repair loop 40 extending across a surface of the tissue 34 and the bone 32, the repair construct 10 is nearly fully tensioned. By further pulling on the first end 18a of the third section 18, the tissue repair construct 10 will be fully tensioned and a taut repair loop 40 will be formed to compress the tissue 34 to the bone 32. The finger trap configuration of the spliced region 24 may allow the repair loop 40 to be tightened, but not loosened. Once the tissue repair construct 10 is fully tensioned, the user may trim away and discard any portions of the third section 18 that are not part of the spliced region 24 or immediately adjacent to the spliced region 24. FIG. 4F shows the completed repair with four constructs 10 being used with four tunnels 36. However, the disclosure contemplates that more or fewer than four tunnels 36 and/or constructs 10 could be used to complete the repair in a variety of routing geometries.

While the disclosure particularly shows and describes preferred examples, those skilled in the art will understand that various changes in form and details may exist without departing from the spirit and scope of the present application as defined by the appended claims. The scope of this present application intends to cover such variations. As such, the foregoing description of examples of the present application does not intend to limit the full scope conveyed by the appended claims.

I claim:

1. A tissue repair construct comprising:
a repair suture, the repair suture comprising:
a first section;
a second section connected to the first section; and
a third section connected to the second section opposite the first section, the third section comprising a spliced region adjacent to the second section; and
a transfer suture, a portion of the transfer suture extending through the spliced region such that first and second ends of the transfer suture extend from opposite ends of the spliced region;
wherein the spliced region forms a finger trap, such that the spliced region allows movement of the transfer suture through the spliced region in a first direction, but prevents movement of the transfer suture through the spliced region in a second direction opposite the first direction.

2. The tissue repair construct of claim 1, wherein a material of the second section is selected to be different than a material of the first section.

3. The tissue repair construct of claim 1, wherein a material of the third section is selected to be different than a material of the first section.

4. The tissue repair construct of claim 1, wherein a material of the third section is selected to be different than a material of the second section.

5. The tissue repair construct of claim 1, wherein the first section comprises 2-0 suture.

6. The tissue repair construct of claim 1, wherein the second section comprises suture tape.

7. The tissue repair construct of claim 1, wherein the third section comprises No. 2 suture.

8. The tissue repair construct of claim 1, wherein the first end of the transfer suture includes a transfer loop.

9. A tissue repair construct comprising:
a repair suture, the repair suture comprising:
a first section;
a second section connected to the first section; and
a third section connected to the second section opposite the first section, the third section comprising a spliced region adjacent to the second section;
wherein the first section and a portion of the second section extends through the spliced region, forming a repair loop; and
wherein the spliced region forms a finger trap, such that the repair loop can be tightened around a repair but not loosened.

10. The tissue repair construct of claim 9, wherein a material of the second section is selected to be different than a material of the first section.

11. The tissue repair construct of claim 9, wherein a material of the third section is selected to be different than a material of the first section.

12. The tissue repair construct of claim 9, wherein a material of the third section is selected to be different than a material of the second section.

13. The tissue repair construct of claim 9, wherein the first section comprises 2-0 suture.

14. The tissue repair construct of claim 9, wherein the second section comprises suture tape.

15. The tissue repair construct of claim 9, wherein the third section comprises No. 2 suture.

16. A method of tissue repair comprising:
forming a tunnel through a bone adjacent a tissue in need of repair;
passing a tissue repair construct through the tunnel and through the tissue such that a first end of a third section and a second end of a transfer suture of the tissue repair construct extend from the tunnel, and a first section and a first end of the transfer suture of the tissue repair construct extend from of the tissue, the first end of the transfer suture including a transfer loop, the tissue repair construct further comprising:
a repair suture, the repair suture comprising:
the first section;
a second section connected to the first section; and
the third section connected to the second section opposite the first section, the third section comprising a spliced region adjacent to the second section;
wherein a portion of the transfer suture extends through the spliced region such that the first and second ends of the transfer suture extend from opposite ends of the spliced region;
passing the first section of the tissue repair construct through the transfer loop;
using the second end of the transfer suture, pulling the first section and a portion of the second section through the spliced region to form a repair loop extending along a surface of the tissue and the bone; and
tensioning the repair loop to secure the tissue against the bone;
wherein the spliced region forms a finger trap, such that the repair loop can be tightened along the surface of the tissue and the bone, but not loosened.

17. The method of claim 16, wherein the bone is a humeral head.

18. The method of claim 16, wherein the tissue is a rotator cuff.

19. The method of claim 16, wherein the transfer suture is preassembled to the repair suture.

20. The method of claim 16, wherein the second section comprises suture tape.
